(19) United States
(12) Patent Application Publication
(10) Pub. No.: US 2007/0272746 A1
(43) Pub. Date: Nov. 29, 2007

(54) Medical Device Radio Frequency Identification System and Method

(76) Inventors: Bennett Ortiz, Miami, FL (US); Norman Sandberg, Haifa (IL)

Correspondence Address: Philip S. Johnson;Johnson & Johnson, One Johnson & Johnson Plaza, New Brunswick, NJ, 08933-7003, US

(21) Appl. No.: 10/579,517

(22) PCT Filed: Nov. 23, 2004
(86) PCT No.: PCT/US04/39401

§ 371 Date: Apr. 19, 2007

Related U.S. Application Data
(60) Provisional application No. 60/525,227, filed on Nov. 25, 2003.

(51) Int. Cl. G06Q 50/00

(52) U.S. Cl. 235/385; 705/2; 705/28

(57) ABSTRACT

A medical device inventory management system, including one or more radiofrequency identification (RFID) transponder or “tag” associated with one or more medical devices or attached to the medical device packaging. One or more RFID readers may be used, which may take the form of a handheld device or a shelf or shelving system for holding medical devices, or a handheld device may be used in conjunction with a shelving system. The RFID reader communicates automatically with a computer or computer network, providing such information as inventory status, whether a specific medical device is present in inventory, inventory amount of medical devices, etc.

### CROSS-REFERENCE To RELATED APPLICATION

This application claims the benefit of priority of U.S. Provisional Patent Application No. 60/525,227 filed on Nov. 25, 2003.

### BACKGROUND AND SUMMARY OF THE INVENTION

1. Technical Background

The present invention relates to a system for medical devices using radiofrequency identification, and more particularly to a medical device inventory management system using radiofrequency identification.

2. Discussion

Radiofrequency identification (RFID) systems are used for a variety of different purposes. Some existing applications include paying for purchases (for example, Mobil SpeedPass®), product theft prevention (for example, RFID devices at the entrances of many retail stores), bar-code replacement systems, and secure access to a building using a personal RFID security badge. RFID systems have been described as a kind of radiofrequency “smart bar-code.”

RFID systems have also been used for item tracking systems, and “real-time” inventory management. For example, U.S. Pat. No. 6,681,990, entitled “Item Tracking Systems and Real-Time Inventory Management” which issued to Vogler et al. on Jan. 27, 2004, describes a real-time inventory management system in which “a monitoring system monitors the state of tagged items located within an inventory and sends an event to an event router when an item is added to or removed from the inventory. The event router receives the event from the monitoring system and sends the event to one or more item tracking systems. The item tracking systems receive the event and update stored information about the item to reflect the event.” (Column 1, lines 37-44.)

In a retail location, for example, an item tracking system may be part of an inventory management system used at a retail store, which has inventory having RFID tags or transponders. When tagged items are enter or leave the store, an RFID monitoring system will note these “inventory events.” Alternately, when tagged items are removed from the shelf, or are replaced on the shelf, the RFID system will record these events. Also, the item tracking system may obtain alerts when an inventory level of a certain product falls below a certain amount, indicating that the product should perhaps be replenished.

There are two types of RFID tags: passive and active. A passive tag has no power source for communications or data transmission, while an active tag has some kind of internal power source such as a battery. An active tag may also have some computing or processing capacity.

RFID tags are generally capable of being electronically initialized, and storing a digital identification code, which can be read directly from the tag using an RFID reader. Some tags are capable of holding much more information, and some are re-writable.

As far as the RFID reader, it may include a frame or housing, one or more antennas, a radiofrequency interrogator, a radiofrequency multiplexer, and a computing system. It may be provided with software of varying sophistication.

The products used, and the packaging in which they are contained, may affect performance of the RFID system. Medical devices, for example, may present additional challenges in using an RFID inventory management system.

For instance, medical devices often have a specific expiration date, and it would be desirable to enable an RFID inventory management system to automatically provide or communicate an alert when a medical device has expired, or when it will do so within a certain period.

A medical device RFID inventory management system may also encourage health care professionals to use “first-in, first-out” procedures, referred to as “FIFO,” by providing an immediate alert that a medical device removed from a shelf or inventory area(s) has a later expiration date than another medical device on that shelf or in an inventory area. This alert gives the health care professional the ability to replace that medical device, and instead to use the medical device first which expires first.

Medical devices may also be provided in packaging which includes metal. However, the presence of metal provides challenges in obtaining clear radiofrequency reception, and therefore accurate readings of the identification code initialized on the RFID tag. Overcoming such challenges by designing the arrangement of positioning of the RFID system components may be referred to as “metal immunity.”

Also, medical devices may be temperature-sensitive, so they may be unapproved for use if they are exposed to conditions above a specific temperature.

In an alternate embodiment, the RFID inventory management system may be adapted to associate a patient identification code with the RFID identification codes for those medical products that are used to treat (or are implanted in) that patient. The patient identification code may generally be obtained from a patient wristband, either manually, by bar-code reader, or with another RFID tag embedded in the patient wristband.

The term “inventory” is used in its broadest sense, to encompass any object(s) or merchandise in one or more locations, or within an area of responsibility or control, and specifically includes items on consignment.

RFID inventory management systems for medical devices may provide several advantages, which may include automatic (or passive) reporting of inventory amounts, automatically updated inventory information or reports based on the presence of individually or collectively tagged medical devices, and/or their presence, removal and/or replacement of tagged medical devices within an inventory area.

Automatic alerts may be provided, based on any of various criteria, such as preselected minimum desired inventory amounts or replenishment levels, or expiration of certain medical device(s) in inventory, or future medical device expiration within a specified period of time. If a medical device tag is also provided with a temperature sensor, an automatic alert may be sent if a selected temperature is exceeded.

A medical device RFID inventory management system may also encourage health care professionals to use “first-in, first-out” procedures, by immediately alerting them that one of the medical device(s) on a shelf or in an inventory area(s) has an earlier expiration date than the one that was just pulled for use.

In addition, all aspects of a medical device RFID inventory management system, including the timing and detail of inventory reporting and/or alerts, can be customized to particular types of medical devices, or to particular locations, customers, or hospitals.

It is also possible that a medical device RFID inventory management system may coordinate inventory amounts in various locations, even for example among different hospitals. If an inventory amount of a certain medical device falls below at preselected amount (or is soon to expire), and a nearby location has an excess number of that product (or which expire at a later date), an RFID inventory management system may provide an alert that allows inventory to be traded among the different locations.

It should be noted that the present invention relates not only to RFID medical device inventory management systems, but also the components of such systems, and methods for using them.

These and various other objects, advantages and features of the invention will become apparent from the following description and claims, when considered in conjunction with the appended drawings. The invention will be explained in greater detail below with reference to the attached drawings of a number of examples of embodiments of the present invention.

### BRIEF DESCRIPTION OF THE DRAWINGS

FIG. 1 is a partial perspective view of medical device packaging, with an RFID transponder on the spine of the packaging;

FIG. 2 is a partial perspective view of medical device packaging, with an RFID transponder on the face of the packaging;

FIG. 3 is a schematic drawing of an RFID transponder;

FIG. 4 is a front elevation view of a shelving system for medical devices;

FIG. 5 is a rear elevation view of the shelving system of FIG. 4;

FIG. 6 is a perspective view of a handheld device;

FIG. 7 is a diagrammatic drawing of an example patient wristband;

FIG. 8 is a perspective view of a handheld device, in use with medical device packaging having an RFID transponder;

FIG. 9 is a perspective view of a handheld device, in use with a patient wearing a wristband; and

FIG. 10 is a diagrammatic drawing of one example of a medical device RFID inventory management system.

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

The drawings depict examples of medical device RFID inventory management systems and components arranged according to the principles of the present invention. However, it should be noted that a wide variety of medical device RFID inventory management systems and components may be made, which incorporate the claimed feature of the present invention.

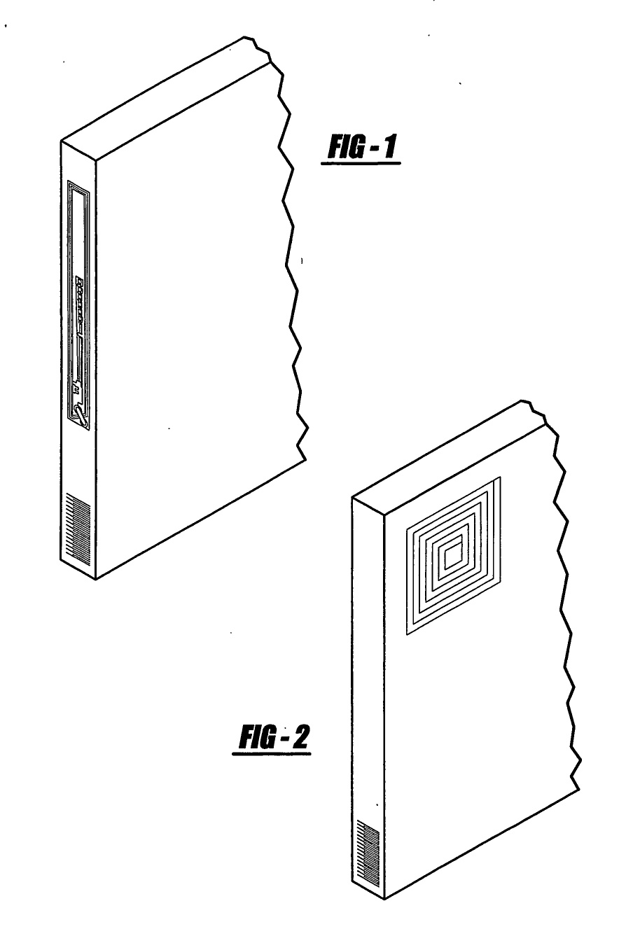

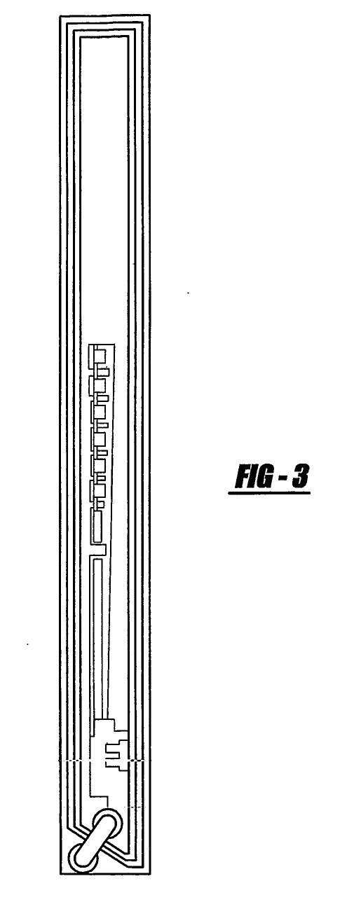

Examples of medical device packaging are depicted in FIGS. 1 and 2. If desired, the product packaging may include a bar-code, as shown. Of course, such a bar-code is optional for purposes of a medical device RFID inventory management system according to the present invention. An RFID “tag” or transponder is also affixed to each package. In FIG. 2, a diagrammatic view of a conventional type of RFID tag is shown, affixed to the larger face of the package. In contrast, FIG. 1 shows a novel type of RFID tag, specially designed to be affixed to the package spine.

The shape of an RFID tag has not previously been considered important, so long as it fits on or in the packaging. However, some medical devices are metal, or are packaged in metal, for example a metal foil inner pouch.

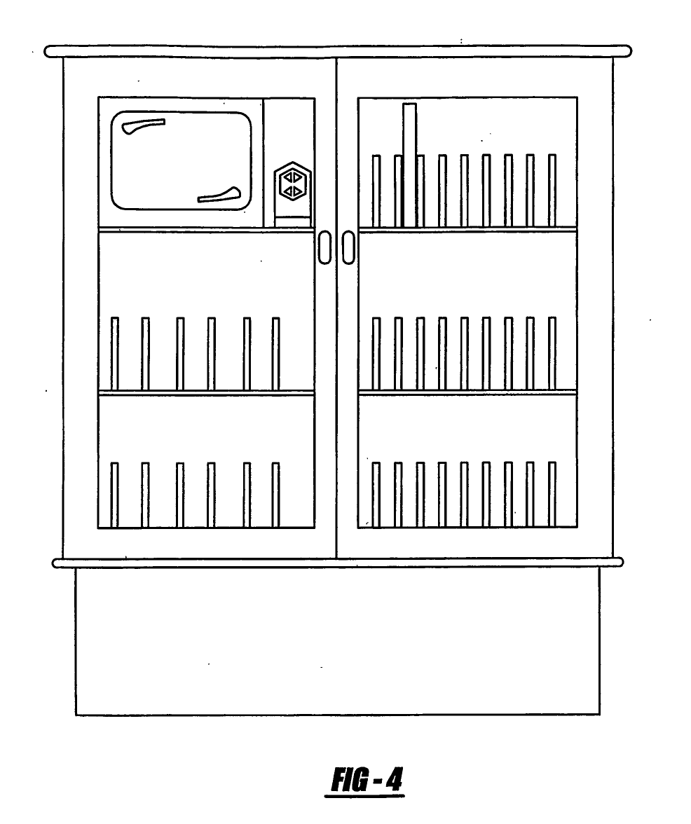

In this case, the novel type of “narrow” RFID tag shown in FIG. 1, which allows placement on the spine of the package, will enhance the performance and accuracy of a RFID inventory management system. FIG. 3 shows a more detailed depiction of such a “narrow” RFID tag.

In FIG. 1, the plane of the tag is perpendicular to a majority of the metal in the medical device and packaging. The resulting enhancement characteristic may be referred to as “metal immunity.”

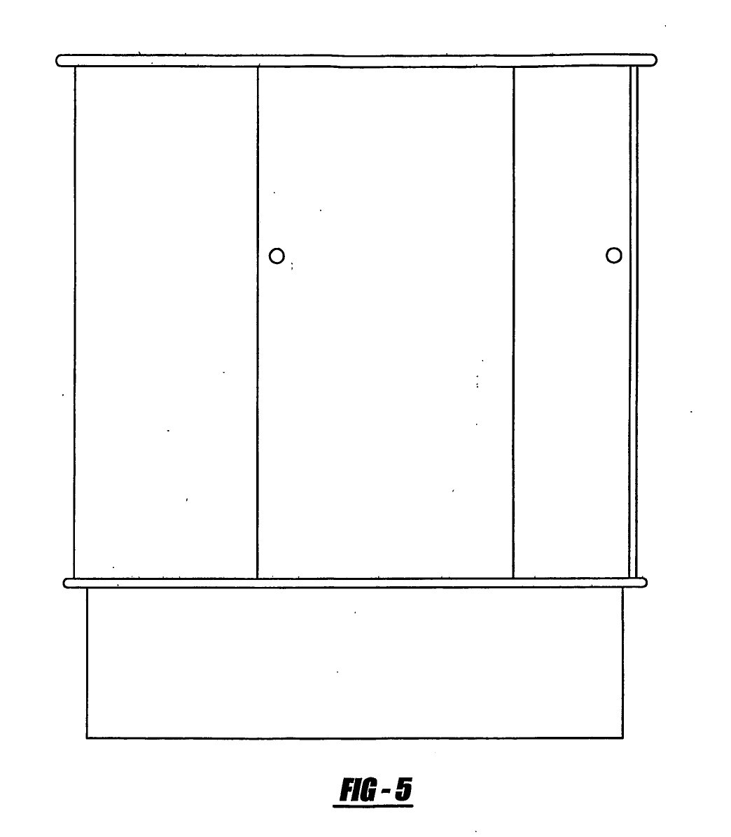

FIGS. 4 and 5 show an example of a “smart shelf” unit, viewed from the front and rear, respectively. This particular example has several shelves, and dividers for causing individual medical device packages to stand upright. Only one medical device is shown in FIG. 4, but the shelves of course may hold any number of medical devices at a time. The smart shelf includes antenna(s) and other RFID components, so that the entire unit is an RFID reader.

An optional touch-screen computer is also shown in FIG. 4, so as to provide a direct interface for a health care professional to the RFID inventory management system. In addition, FIG. 4 shows an optional bar-code reader, which allows bar-code information to be added to the medical device inventory management system.

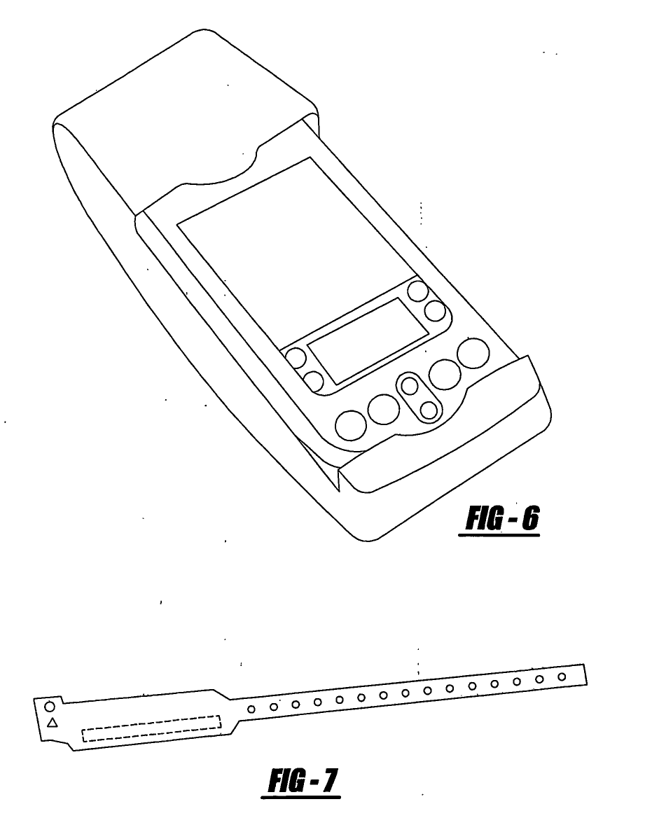

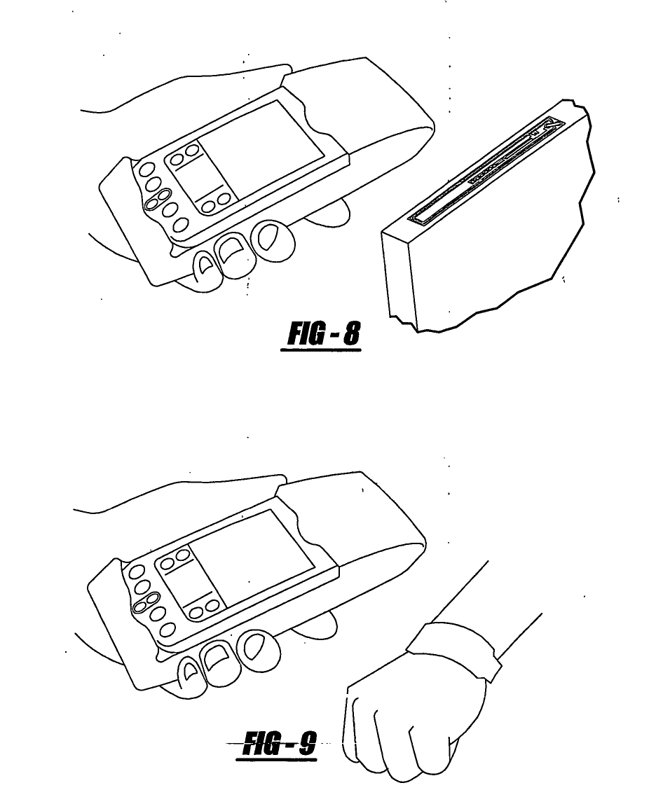

An example of a handheld RFID reader is shown in FIG. 6. In this example, a readily-available handheld computer has been equipped with a specialized cradle. The cradle includes the various RFID hardware, including antenna(s) and an RF interrogator. A handheld RFID reader is shown in FIGS. 8 and 9, being used to read an RFID tag on a medical device package in FIG. 8, and a patient wristband in FIG. 9.

Each of the RFID readers communicates with the main database of the inventory management system, either wirelessly, by e-mail, through an ethernet or direct internet connection, etc. Accordingly, one or more handheld RFID readers and one or more of the “smart shelves” may be used independently, or together.

An example of a patient wristband is shown in FIG. 7. The patient wristband may have a bar-code or an additional RFID tag.

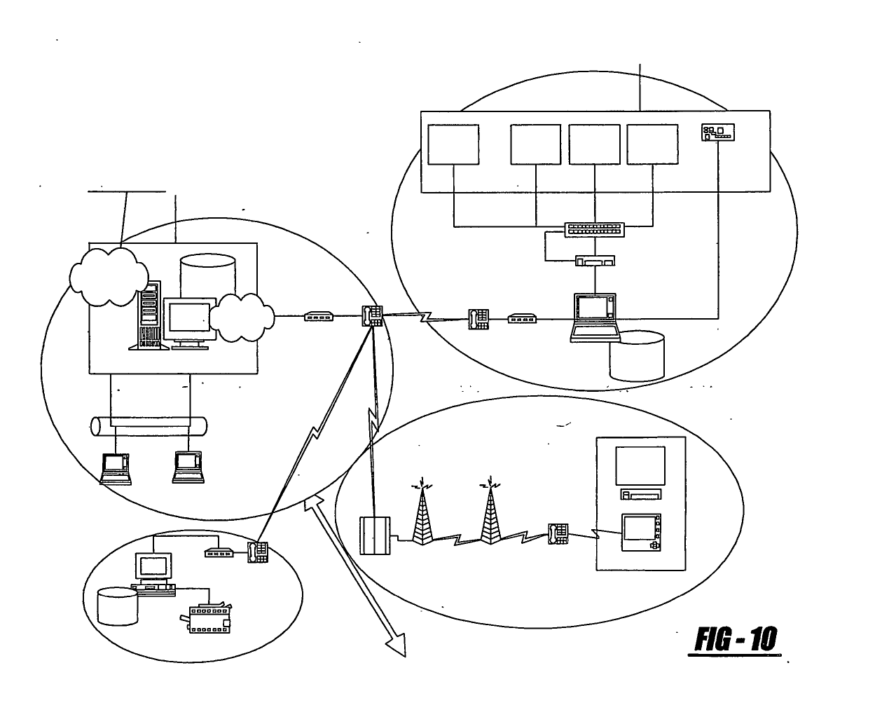

A diagrammatic depiction of a medical device RFID inventory management system is shown in FIG. 10, including an initialization site for initializing RFID tags to identification codes, one or more local sites where medical device inventory is to be used (a hospital, for example), a remote site where the main database is maintained, and optionally one or more handheld units.

Local (Hospital) Site:

The local site may be enabled for monitoring the inventory levels of RFID-enabled products, possibly with the addition of monitoring thermal conditions, at a predetermined frequency. It may be also enabled for transmitting all acquired data back to an administrator for further analysis and subsequent business actions. The local data cache (local database) is updated accordingly to reflect inventory events and maintain inventory history. The site is located on the premises of a hospital or mini distribution center and may contain several components, as follows.

RFLD Subsystem (Smart Shelf):

The REID Subsystem (Smart Shelf) may include several different types of components, which may include the following: a frame, a radiofrequency (RF) antenna or antenna group, RF signal cable(s), an RF multiplexer, an RF interrogator, control cable(s) for the RF interrogator, and a shelf or shelving system for medical devices inventory.

Computer Controller:

The computer controller may include several different types of components and accessories, which may include the following: a personal computer, which may be a laptop computer, interface hardware, communication hardware, and operation system software.

Communication Link:

The communication link may include several different types of components, which may include the following: telephone line and/or Ethernet connection.

RFID Subsystem (Smart Shelf):

The RFID subsystem or “Smart Shelf” is capable of providing automatic inventory information acquisition. The antennas, discreetly placed within the shelf unit, are connected to the RF multiplexer via cables, and are positioned in such a way so as to permit the signal acquisition of all medical device inventory units placed upon the shelf.

In addition, the antennas may be positioned so as to enable and facilitate signal acquisition, regardless of metal content that may be in the medical device or in the packaging, an arrangement referred to as “metal immunity.” The multiplexer determines which antenna is selected for the RF interrogator. The software may be also so designed as to acquire and negate any duplicate signal acquisitions. The interrogator and the multiplexer are also controlled by the software.

Sensors may also be installed so as to monitor ambient environmental conditions. These sensors will also be controlled by the software. In addition, the “smart” shelf may be secured through RF technology by associating access system rights to the inventory data, or by an encryption system.

Controller Computer:

The controller computer executes the software, as well as holds the local data cache. The local data cache records current inventory and the history of the individual products. The local data cache may also possibly include data regarding ambient environmental conditions, including for example temperature and humidity.

It may be possible to enable or maintain local operation of the inventory management system, in case the communication link between the local site and the remote site become unavailable or unreliable.

Each medical device or product or its packaging can carry an initialized RFID transponder or “tag.” The transponder may be strategically positioned so as to minimize or eliminate any interference that may be due to the presence of metal.

The software provides a user interface for the system, which may include predefined queries and inventory reports. It also may generate automatic inventory notifications such as: replenishment needs, billing details, and product expiration notifications. In addition, it may enable the connection and automatic updates to the remote site (master database) at predetermined time intervals or on demand, and may have a function for associating each pulled product with the medical identification number of the patient receiving it. The master database is the central repository for any and all inventory events.

The user interface is intended to assist the hospital in its inventory assessment functions, as well as to notify users of shortages, expired products, and to assist them in the re-ordering of any products which they may need. It can also be used to register and associate the medical identification of the patient receiving the particular product being removed from the shelf. It may also enable the following one or more of the following features:

1. View current inventory levels as registered during the last inventory scan.

2. Conduct an inventory scan on demand, as opposed to a previously scheduled interval.

3. Start and stop an automatic inventory scan cycle.

4. Adjust the time interval (frequency) of inventory scan cycles.

5. Enter medical record identification number or patient identification code, associated with individual product(s).

6. To configure and establish on demand communication link between the local site and the remote site;

7. Conduct on-demand data exchange between the local site and the remote site.

8. View inventory notifications and information.

9. Automatically generate e-mails or alerts to predetermined individuals and to an administrator and/or sales representatives, regarding variances in recommended inventory levels or pending product expiration.

10. View product expiration reports.

11. View items which may have fallen below predetermined inventory levels.

12. View product information transmitted from and supplied by an administrator.

Communication Link:

At least three communication link options are possible: a direct internet connection to the remote site, a dial-up connection to the remote site, or a wireless connection which may be made using “Wi-Fi” and/or “Bluetooth” technology.

In each of these types of communication links, once the link is established the communication protocol will generally be that of the world-wide web: HTTP or HTTPS over TCP/IP. The software protocol between the client and the server software may be SOAP, and the server may implement J2EE web services.

Remote (Server) Site:

The remote site could be located at a site of the medical device manufacturer or another campus. Its main function is to provide centralized data repository for all local (Hospital) sites. In other words, this is the master database. In addition, the remote site provides user interface, which allows the following operations which can be executed by an administrator:

1. View all automatically generated e-mails or alerts, warning of inventory levels below recommended levels.

2. Generate, view and print product use or inventory reports, by local site or system-wide.

3. Generate, view and print last-known inventory reports, per local site or system-wide.

4. Generate, view and print summary inventory reports.

5. Generate, view and print summary inventory usage reports.

6. Generate, view and prim product expiration reports.

7. Generate, view and print product history reports.

8. Generate an interface file which can be used for a data exchange with the billing system of the medical device manufacturer.

9. Generate an interface file that can be used with the order management system of the medical device manufacturer. This feature can allow an administrator to communicate with the transponders on the medical device inventory, indicating expiration status as well as multiple other events. In effect, the inventory can then alert against its own use, or notify the master database to perform immediate replenishment.

10. Configure queries and reports, which can provide any and all permutations of the available data.

The remote site should include either a direct internet connection, or dial-in modem to allow the hospital site and an administrator to connect on demand and replicate data. The software may implement J2EE compliant web service, which can be the entry point for the local site and may be responsible for direct update of the master database with inventory events.

Initialization Site:

The initialization site initializes and assigns RFID transponders to the individual medical devices or products. This may occur at the manufacturing site designated for final packaging and labeling, or some other designated site. The information initialized to the transponder may include, but is not limited to: a unique identification code, product labeling, and/or traceability information.

The initialization site may include the following components: controller CPU, software, RFID and/or bar-code printer.

The controller computer executes the software. The software may be integrated into the medical device manufacturer's packaging software and ERP systems, but may also provide a user interface to allow the user to select product type and the number of RFID transponders they want to produce and initialize. In addition, the computer registers the newly produced transponders with the master database at the remote site. The RFID barcode-type printer both prints and initializes the new transponders, which are then affixed to the product by manufacturing personnel.

There are at least two options to connect the initialization site with the remote site. If both sites are located at the same campus, a direct ethernet connection can be made. Or if the initialization site is geographically removed from the remote site, a direct internet connection or dial-up connection can be established. In each case, the data updates would be done via the web service mentioned above.

Inventory Administration:

An administrator or administration organization may be empowered to review and act upon the various inventory reports which are either manually or automatically generated. The administrator may also be the initialization site for all inventory notifications which for any reason need to be communicated to the product transponders at the local sites. The administrator may analyze the information, and may act upon it according to selected parameters.

Point-of-use information may be acquired and used to determine inventory levels as well as to generate accurate demand forecasts. It may be used to predict product expiration and variances in recommended levels of inventory. These reports and information may be integrated into the medical device manufacturer's ERP systems, depending upon the efficacy and usefulness of the information.

The Handheld:

The handheld unit may provide a quicker and more economic way of capturing inventory information, without the need for introducing relatively bulky RFID shelves to the local site. All of the informational functionality of the smart shelf solution is applicable to the handheld without the need of placing the full RFID smart shelf unit in place.

The handheld device may replace the smart shelf at a particular local hospital site, with all other interfaces unchanged. The handheld device may be used to scan the products, and then communicate the acquired information to the master database at the remote site. The handheld may be based on the following components: an RFID-enabled handheld computer, with a modem and/or wireless interface, and software.

The handheld executes the software, which controls an RFID interrogator, which may be built into the handheld device. It also may provide a graphical interface to allow the following operations:

1. Selecting the location at which inventory is being taken from a list of known locations. If the location is new, the software may allows it to be added to the list of locations. For local sites or hospitals with multiple locations, an RFID transponder may identify each location uniquely.

2. Start or stop an RFID scanning cycle.

3. Accept or cancel the results of an RFID scanning cycle.

4. Establish dial-up or wireless connection to the remote site and upload inventory levels and inventory events to the master database.

Any data replication with the master database may be done via a web service in the same manner as with the smart shelf unit. In other words, the handheld system may be programmed to register inventory and inventory events as are possible with the smart shelf unit.

Operational Scenarios:

The following is a description of one possible method of using an RFID system to manage medical device inventory:

1. Product is packaged, labeled and affixed with a RFID transponder at a manufacturing site of choice. The data transfixed on the transponder and associated with the respective Product is communicated to the remote site and the master database.

2. Product is shipped to a Local Site (Hospital). If the local site is equipped with RFID shelf, and that shelf is configured to automatically scam for inventory, it can detect the any new products and register the inventory events with its local data cache.

3. At a predefined interval of time, the local site can establish a communication link and update the master database with those inventory events. The information for each product and its history will be modified to reflect its removal from the shelf (use) and other status.

4. Optionally, and in addition to monitoring of the inventory levels, the system may monitor the ambient environmental conditions at each cycle. This information may be also recorded and automatic warnings issued if the temperature level becomes critical. If the environmental conditions are compromised, the products could be flagged for replacement.

5. In addition to this, regular e-mails may be sent to an administrator and Sales Representatives, issuing notification of low levels of inventory and possible impending product expiration.

6. when product(s) are used a qualified operator can enter the medical record ID and remove the products from the shelf. This operation may cause the medical record ID to be associated with the product(s) used. If products are returned back, the system will detect them and de-associate them from the medical record. Any changes however will be recorded and dated in the product's history records.

7. If the local site is not equipped with RFID shelf, the operator can use the handheld solution to scan the inventory in storage at will. Later he or she may establish communication with the remote site to upload the results of this scan to the master database.

8. At the remote site the Web Service will execute and await inventory updates and apply them appropriately to the master database.

9. At predefined intervals of time, the software will query the master database to determine what products should to be replenished per location, based on the last known inventory and the requested levels for that location. The software then will automatically create order reports as necessary. These order reports may then be accessed by an administrator or the Local Site (Hospital).

10. In addition to the queries and reports the master data base when advised of a product shortage can suggest appropriate substitutions and transfer this information to the hospital, an administrator, and via e-mail to the appropriate sales representative.

11. This remote site may also feature a number of web-based reports (as described above). Those may be based on the inventory and order information found in the master database and will help organize billing and orders as well as to increase product visibility. Additional web-based user interfaces are possible and can configured as required.

12. An administrator may use the intelligence offered by the system in order to determine better service levels to the customer, and to ensure that the right patient is getting the right product at the right time.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. a medical device inventory management system, comprising:
a medical device defining an expiration date, in medical device packaging;
an RFID transponder affixed to the medical device packaging; the RFID transponder being initialized with an identification code;
an RFID reader defining a proximity distance at which an RFID transponder is readable, adapted to generate and receive wireless signals to and from an RFID transponder, thereby reading the identification number from the RFID transponder;
a computer having memory loaded with software;
such that when the medical device is in proximity to the RFID reader, the RFID reader interrogates the RFID transponder to read the identification code into the computer memory; and when the medical device is removed from proximity to the RFID reader, the RFID reader communicates the removal of that identification code to the computer memory; and if the medical device is again placed in proximity to the RFID reader, the RFID reader will again interrogate the RFID transponder to read the identification code into the computer memory;
wherein the computer software is adapted to periodically communicate all identification codes of medical device RFID transponders in proximity to the RFID reader to an inventory administrator; and to automatically communicate an alert to the inventory administrator if an inventory of medical devices is reduced below a preselected amount; and to automatically communicate an alert to the inventory administrator if a medical device has reached the expiration date or is within a preselected time period of the expiration date.

2. The medical device inventory management system of claim 1, further comprising a bar-code reader.

3. The medical device inventory management system of claim 1, further comprising a patient wristband reader.

4. The medical device inventory management system of claim 1, wherein the medical device packaging is at least partially metal, wherein the RFID tag has a shape that maximizes the accuracy of the RFID reader.

5. The medical device inventory management system of claim 1, wherein the medical device packaging further comprises a temperature sensor, and wherein the RFID reader is adapted to also interrogate the temperature sensor.
